(19) United States
(12) Patent Application Publication
(10) Pub. No.: US 2021/0280313 A1
(43) Pub. Date: Sep. 9, 2021

(54) POLYGENIC RISK SCORE MODIFIES RISK OF CORONARY ARTERY DISEASE CONFERRED BY LOW-DENSITY LIPOPROTEIN CHOLESTEROL

(71) Applicant: ALLELICA S.r.l., Roma (IT)

(72) Inventors: Giordano BOTTA', Roma (IT); Paolo DI DOMENICO, Roma (IT); Alessandro BOLLI, Roma (IT)

(21) Appl. No.: 17/191,004

(22) Filed: Mar. 3, 2021

Related U.S. Application Data
(60) Provisional application No. 62/985,033, filed on Mar. 4, 2020.

(51) Int. Cl. G16H 50/20; G16B 20/00; G16B 40/00; G06N 20/00; G16H 50/30

(52) U.S. Cl. CPC G16H 50/20; G16B 20/00; G16H 50/30; G06N 20/00; G16B 40/00

(57) ABSTRACT

A method for a predictive prognosis of onset of a cardiovascular disease in an individual acquires/accesses low-density lipoprotein cholesterol level, calculating a risk value and determining an individualized risk value of onset of the cardiovascular disease, based on the calculated risk value. Calculating a risk value includes calculating the risk value representative for onset of the cardiovascular disease, based on operational values of a polygenic risk score and low-density lipoprotein cholesterol level, based on first, second and third risk parameters are representative of risk of onset of: the cardiovascular disease induced by the polygenic risk score, the cardiovascular disease induced by the low-density lipoprotein cholesterol level, and the cardiovascular disease induced by interaction of polygenic risk score and low-density lipoprotein cholesterol level. The risk parameters are calculated based on a pre-trained model and/or algorithm, to which the operational values of polygenic risk score and low-density lipoprotein cholesterol level are input.

### TECHNOLOGICAL BACKGROUND OF THE INVENTION

### Field of Application

The present invention relates to a method for a predictive prognosis of the onset of a cardiovascular disease in an individual.

The general technical field of the present invention is therefore that of predictive methods, performed by electronic computation, used in the medical field to support predictive prognoses.

### Description of the Prior Art

An individual's lifetime risk of Coronary Artery Disease (CAD) is determined by a combination of genetic and lifestyle factors. Whilst adherence to a healthy lifestyle can help individuals with high genetic risk reduce their lifetime risk of CAD, the extent to which blood lipid levels affect CAD risk in individuals with varying genetic risk remains unknown.

Coronary artery disease (CAD) is the leading cause of death worldwide (Lozano et al., 2012). CAD is a complex disease and its etiology is characterized by the complicated interplay between both genetic and lifestyle factors. 2,213 genetic variants have been found to be significantly associated with CAD (Nikpay et al., 2015), highlighting the polygenic architecture of the disease. Effect sizes of risk-associated alleles identified in Genome-Wide Association Studies (GWAS) can be combined to generate a Polygenic Risk Score (PRS), a quantitative metric used to stratify individuals at different polygenic risks (Torkamani et al., 2018). A CAD PRS developed by Khera et al. (2018) identified that 8% of individuals of the UK Biobank have an increased risk of CAD that is more than three times higher than the remainder of the population, which is similar to the risk conferred by familial hypercholesterolemia. In a second study, men in the top 20% of a CAD PRS distribution reached a threshold of 10% cumulative CAD risk by 61 years of age, more than 15 years earlier than men in the bottom 20% of the distribution (Inouye et al., 2018).

CAD PRS can be used in the general population to identify those individuals that would benefit the most from targeted lifestyle recommendations and preventive interventions. A high genetic risk of CAD has been shown to be actionable in two ways. First, individuals at high genetic risk of CAD who adhere to a healthy lifestyle display a two- (Khera et al., 2016) or three-fold (Said et al., 2018) reduction in the relative risk of developing CAD, compared to individuals with a poor lifestyle. Second, in a randomized controlled trial of men with hypercholesterolemia treatment with lipid-lowering medications reduced CAD risk from 19.6% to 11.7%, in individuals in the top quintile of the PRS distribution, and from 12.9% to 10.1%, in the remaining individuals despite both groups had same LDL level at baseline (LDL-C 192 mg/dL), and achieved almost identical LDL levels after treatment (148 and 149 mg/dL, High PRS and remainder, respectively). This finding suggests that the polygenic background may act as an enhancer, increasing CAD risk by facilitating the LDL-C-dependent process of plaque formation (Ference et al., 2018).

Despite such evidence, a systematic assessment of the interplay between polygenic risk and blood lipids in contributing to the overall CAD risk is still lacking. To address this, we ask the following questions: i) How does the PRS odds ratio range compare to those of blood lipids commonly used to estimate CAD risk? ii) Is PRS correlated to conventional risk factors? iii) Is CAD risk conferred by blood lipids modulated by the polygenic background? iv) If and to what extent optimal blood lipid levels are associated with reduced CAD risk in individuals with High PRS?

### SUMMARY OF THE INVENTION

It is the object of the present invention to provide method for a predictive prognosis of the onset of a cardiovascular disease in an individual, which allows to obviate at least partially the drawbacks mentioned above with reference to the known art, and to respond to the aforementioned needs particularly felt in the technical field considered. Such an object is achieved by a method according to claim 1.

Further embodiments of such a method are defined by dependent claims 2-12.

It is also an object of the present invention to provide a device adapted to perform a predictive prognosis of the onset of a cardiovascular disease, according to the above mentioned method. Such an object is achieved by a device according to claim 13.

A further object of the present invention is to provide a method for a clinical evaluation or a therapeutic intervention, wherein said method is performed on the basis of the results of the above mentioned predictive prognosis method. Such an object is achieved by a device according to claim 14.

### BRIEF DESCRIPTION OF THE DRAWINGS

Further features and advantages of the method according to the invention will become apparent from the following description of preferred embodiments, given by way of non-limiting example, with reference to the accompanying drawings, in which:

FIG. 1 is a flowchart describing the different sub-populations from UK Biobank used in the different steps of the analyses of the present method; numbers in brackets represent UK Biobank data fields used for filtering;

FIG. 2 illustrates a correlation matrix and odds ratio for incident CAD (Coronary Artery Disease) by PRS (Polygenic Risk Score), lipids and their ratios;

FIG. 3 (including panels A to E) illustrates a predicted risk of CAD events in the UK Biobank prospective cohort according to polygenic risk and blood lipid levels;

FIG. 4 illustrates odds ratio for incident CAD by apolipoproteins;

FIG. 5 shows a correlation matrix between CAD risk factors;

FIG. 6 is a diagram illustrating differential increased CAD risk by LDL-C in groups with different polygenic risk;

FIG. 7 is a diagram illustrating CAD risk by HDL in groups with different polygenic risk.

### DETAILED DESCRIPTION

A method for a predictive prognosis of the onset of a cardiovascular disease in an individual is here described.

The method comprises the steps of acquiring or accessing data comprising the individual's low-density lipoprotein cholesterol level LDL-Cp, calculating at least one risk value R and finally determining and providing, as prognostic result, an individualized risk value Rp of onset of the cardiovascular disease in an individual to be examined, based on the at least one calculated risk value R.

The step of calculating at least one risk value comprises calculating the at least one risk value R, representative of a risk of onset of the cardiovascular disease in the individual, on the basis of an operational value of a polygenic risk score PRS of the individual and an operational value of the individual's low-density lipoprotein cholesterol level LDL-C, and also on the basis of a first risk parameter HR1 representative of a risk of onset of the cardiovascular disease induced by the polygenic risk score PRS, of a second risk parameter HR2 representative of a risk of onset of the cardiovascular disease induced by the low-density lipoprotein cholesterol level LDL-C, and of a third risk parameter HR3 representative of a risk of onset of the cardiovascular disease induced by the interaction of polygenic risk score PRS and low-density lipoprotein cholesterol level LDL-C.

The aforesaid first risk parameter HR1, second risk parameter HR2 and third risk parameter HR3 are calculated on the basis of a pre-trained model and/or algorithm, to which the operational value of polygenic risk score PRS and operational value of low-density lipoprotein cholesterol level LDL-C are provided in input.

Moreover, the step of calculating at least one risk value R comprises calculating a risk value R considering as the operational value of polygenic risk score PRS the value PRSp of the individual's polygenic risk score and as the operational value of the low-density lipoprotein cholesterol level LDL-C the individual's low-density lipoprotein cholesterol level LDL-Cp.

The individualized risk value (Rp) of onset of cardiovascular disease, provided as a result, corresponds to the aforesaid calculated risk value (R).

According to an embodiment of the method, the pre-trained model and/or algorithm comprises an algorithm trained in a preliminary training step, which includes training the algorithm by “machine learning” and/or artificial intelligence techniques, on the basis of known data.

According to an embodiment of the method, the pre-trained model and/or algorithm comprises a regression model, which uses as covariates the polygenic risk score PRS, the low-density lipoprotein cholesterol level LDL-C, and the combination of polygenic risk score and low-density lipoprotein cholesterol level PRS×LDL-C.

In this case, the preliminary training step comprises training the regression model using as a reference dataset a first dataset of a population of individuals of which PRS and LDL-C are known, and for which the outcomes of onset of cardiovascular disease are known, for a first subset of individuals (i.e., cases), or for which the non-onset of cardiovascular disease is known, after a predefined period of time, for a second subset of individuals (i.e., controls).

According to an implementation option, the pre-trained regression model is used to derive the aforesaid first risk parameter HR1, second risk parameter HR2 and third risk parameter HR3.

In this case, the preliminary training step comprises training the regression model with reference to the first risk parameter HR1, second risk parameter HR2 and third risk parameter HR3 on the basis of said reference dataset using as a dependent variable the cardiovascular disease onset or non-onset, obtainable from known data.

According to another implementation option, the pre-trained regression model is used to calculate said at least one risk value R.

In this case, the preliminary training step comprises training the regression model with reference to the at least one risk value R on the basis of the reference dataset using as the dependent variable the cardiovascular disease onset or non-onset, obtainable from known data.

According to an implementation option, the above mentioned regression model is a Cox proportional hazards regression model.

According to an embodiment, the method is adapted to perform a predictive prognosis which takes into account one or more further risk factors.

In this case, said step of calculating at least one risk value R comprises calculating the at least one risk value R also on the basis of an operational value of each of one or more further covariates COVx associated with a respective one of the one or more further risk factors.

The pre-trained model and/or algorithm is configured to calculate the first risk parameter HR1, second risk parameter HR2 and third risk parameter HR3 also on the basis of the operational values of each of the aforesaid one or more further covariates COVx.

In this embodiment, the method further comprises accessing data of the individual to be examined, including individual values COVxp of each of the one or more further covariates associated with the one or more further risk factors.

According to an embodiment of the method, the pre-trained model and/or algorithm comprises a regression model, which uses as covariates, in addition to the polygenic risk score PRS, low-density lipoprotein cholesterol level LDL-C, combination of polygenic risk score and low-density lipoprotein cholesterol level (PRS×LDL-C) variables, also the aforesaid further variables COVx which encode the further risk factors.

In this case, the preliminary training step comprises training the regression model using as a reference dataset a second dataset of a population of individuals of which, in addition to PRS and CDC-L, also the individual values of each of the further covariates COVx is known.

According to an implementation option, the above mentioned further risk factors comprise any sub-set of one or more of the risk factors belonging to the following set:

age, gender, genotyping array, the first 4 principal components of ancestry, Townsend Deprivation Index (TDI), diabetes and smoking status, family history of heart disease, systolic blood pressure (SBP), Body Mass Index (BMI), Glycated hemoglobin, HbA1C, Triglycerides (TG), C-reactive protein (CRP), and dietary intake goals (DIG).

According to an implementation option, the above mentioned logistic regression is adjusted or conditioned on the basis of any sub-set of one or more of the risk factors belonging to the aforesaid set comprising age, gender, genotyping array, the first 4 principal components of ancestry, Townsend Deprivation Index (TDI), diabetes and smoking status, family history of heart disease, systolic blood pressure (SBP), Body Mass Index (BMI), Glycated hemoglobin, HbA1C, Triglycerides (TG), C-reactive protein (CRP), and dietary intake goals (DIG).

According to an embodiment of the method, the risk value R is calculated on the basis of a sum of products relating to different relevant variables, comprising at least the aforesaid polygenic risk score (PRS) variable, low-density lipoprotein cholesterol level (LDL-C) variable, combination of polygenic risk score and low-density lipoprotein cholesterol level (PRS×LDL-C) variable,

According to this embodiment, each product provides the multiplication of the first risk parameter HR1, second risk parameter HR2 and third risk parameter HR3 by the difference between the operational value and the average value of the variables, respectively, polygenic risk score (PRS), low-density lipoprotein cholesterol level (LDL-C), combination of polygenic risk score and low-density lipoprotein cholesterol level (PRS×LDL-C).

According to an implementation option of the method, the determined or calculated risk value is a relative risk value, with respect to an average risk referring to a population,

According to another implementation option of the method, the determined or calculated risk value is an absolute risk value, relating to the individual, and representative of an individual's risk of developing cardiovascular disease within a certain period of time, or at a given age.

According to an application example of the method, the considered cardiovascular disease is a coronary artery disease (CAD).

A device, adapted to perform a predictive prognosis of the onset of a cardiovascular disease, is here described.

The device comprises acquisition means, configured for acquiring a quantity of blood of an individual and determining the individual's low-density lipoprotein cholesterol level (LDL-C) on the basis of a test of the quantity of blood acquired; an electronic interface, adapted to receive in input a calculated value of the individual's polygenic risk score (PRS); and a processor, configured to receive said low-density lipoprotein cholesterol level (LDL-C) and polygenic risk score (PRS) value of the individual, and to perform a method for a predictive prognosis of the onset of cardiovascular disease according to any of the embodiments described above.

The aforesaid acquisition and determination means, electronic interface and processor are contained in a single portable device.

A method for providing a clinical evaluation or for deriving a therapeutic intervention is also comprised in the invention. Such a method is performed on the basis of the results of a predictive prognosis method according to any of any of the embodiments described above.

Further details about a method, according to different embodiments of the present invention, are provided here below, as non-limiting examples.

### Data Sources

The study of the method embodiment, here described, utilizes the UK Biobank population (project number: 40692). Full details of the UK Biobank resource have been described previously (Bycroft et al., 2018). Briefly, between 2006 and 2010, the UK Biobank recruited around 500,000 participants between the ages of 40 and 70 years. At recruitment, information was collected for each participant including physical and functional measurements, demographic characteristics, and self-reported prevalent medical conditions. Incident events throughout the follow-up were recorded and accessed through electronic health care records. Additionally, a wide range of biochemical markers from blood and urine samples was collected at baseline, including risk factors such as blood lipid levels and associated (apo)lipoproteins. Each UK Biobank individual has been genotyped at −850,000 Single Nucleotide Polymorphisms (SNPs) which have subsequently been imputed to over 93 M SNPs.

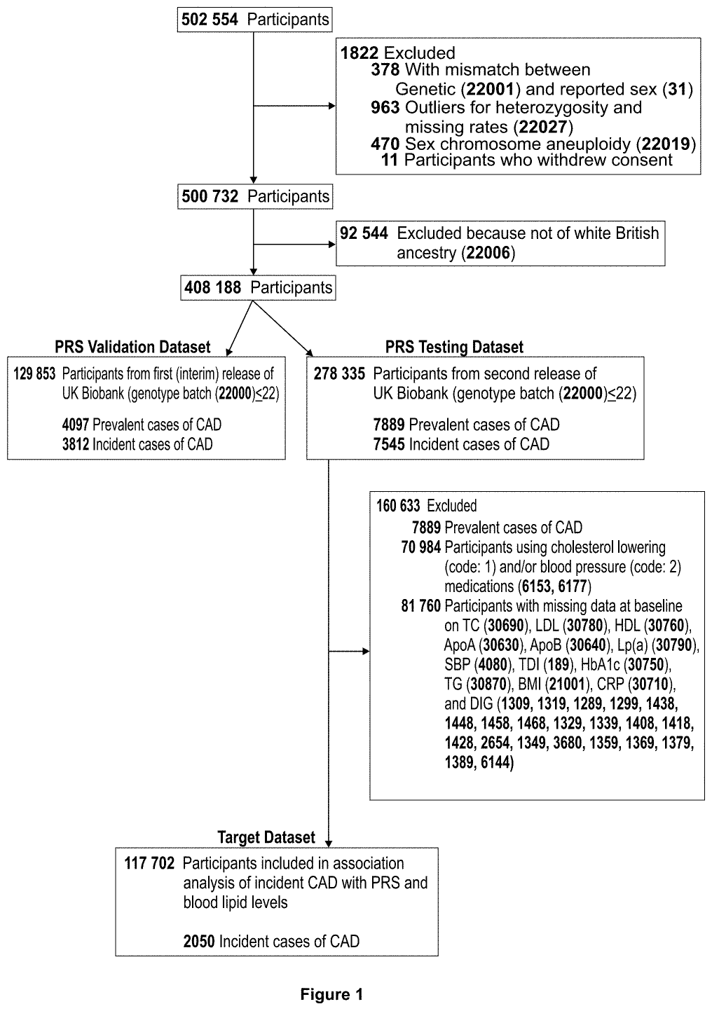

An initial sub-population comprising 408,188 individuals of white British ancestry has been selected. Exclusion criteria comprised the mismatch between genetic and reported sex, outliers in heterozygosity and missing rates, Sex chromosome aneuploidy, and consent withdrawal (as illustrated in FIG. 1).

### Development of CAD PRS

To build a new CAD PRS, in this example the stacked clumping and thresholding (SCT) algorithm developed by Privè et al. (2019) has been applied to GWAS summary statistics of CAD (Nikpay et al., 2015) together with a validation dataset. This led to the identification of a set of SNPs and effect sizes that best predicts the onset of CAD.

The interim release of the UK Biobank has been used as a validation dataset, selecting a total of 129,853 individuals with European ancestry (refer to FIG. 1).

To compare the predictive performance of this new SCT PRS to previously published PRSs, the PRSs have been computed from Khera et al. (2018) and Inouye et al. (2018), and built an additional two novel PRSs by combining the PRS algorithm from Khera et al. with SCT (SCT-K) and Inouye et al. with SCT (SCT-I). the predictive performance of the five PRSs has been assessed by logistic regression and the Area Under the Curve (AUC) of the Receiver Operating Characteristic (ROC) in a testing dataset comprising 278,335 individuals of European ancestry from the second release of UK Biobank (see again FIG. 1).

Logistic regression models used PRSs as predictive variables and presence/absence of a CAD outcome and were adjusted for age at recruitment, gender, genotyping array, and the first four principal components of ancestry.

CAD outcome were defined based on self-reported diagnoses and Hospital Episode Statistics (HES) data (see Table 3).

Prevalent and incident CAD cases were identified by extracting for each individual of the validation and testing datasets the date of the first CAD event. The prevalent and incident conditions were attributed to CAD events that occurred before or after the date of the first UK Biobank assessment, respectively. In order to adhere with and compare to previously published AUC results (Khera et al., 2018), only prevalent CAD cases of the testing dataset were used for AUC calculation.

### Statistical Analysis

Statistical analyses have been performed on a subset of the testing dataset comprising incident CAD cases only (FIG. 1).

Exclusion criteria comprised previous history of CAD (7889), use of cholesterol lowering and/or blood pressure medications (70,984), and missing values at baseline on at least one of the following risk factors (81,760): total (TC), high-density (HDL), and low-density (LDL-C) lipoprotein cholesterol, Apolipoprotein A (ApoA), Apolipoprotein B (ApoB), Lipoprotein a (Lp(a)), systolic blood pressure (SBP), Glycated hemoglobin (HbAlc), Triglycerides (TG), Body Mass Index (BMI), C-reactive protein (CRP), dietary intake goals (DIG) and townsend deprivation index (TDI).

DIG refers to a list of 10 ideal intake goals (Mozaffarian, 2016) comprising the following diet components: Fruit, Vegetables, Whole grains, (Shell)fish, Dairy products, Vegetable oils, Refined grains, Processed meats, Unprocessed meats, and Sugar-sweetened beverages. This led to a target dataset for statistical analysis of 117,702 individuals, comprising 2050 incident CAD cases, with a median follow-up of 8.2 years (5th—percentiles: 6.2-9.4)—see FIG. 1.

Logistic regression was used to determine the ability of PRS and of each lipid risk factor to stratify the risk of incident CAD among individuals of the target dataset. Specifically, the distribution of each risk factor and of PRS was divided into percentiles. Average values of percentiles were used as the predictive variable in logistic regression models to calculate the predicted probability of incident CAD.

Logistic regression models were conditioned on the mean value of additional covariates, namely age at recruitment, gender, genotyping array, the first four principal components of ancestry, SBP, family history of heart disease, smoking and diabetes status, TDI, BMI, HbA1C, TG, CRP, and DIG. Odds ratio per percentile of risk factor and PRS distributions were calculated by dividing predicted percentile-dependent CAD probabilities by the probability of the 50^{th} percentile.

In order to assess the correlation of quantitative CAD risk factors (LDL-C, HDL, TC, TC:HDL, LDL-C:HDL, ApoA, ApoB, ApoA:ApoB, Lp(a), BMI, SBP, HbA1C, TG, CRP) and PRS among individuals of the target dataset, a pairwise correlation matrix was calculated. Specifically for each individual, his corresponding percentile value was calculated in each risk factor distribution. Then, a matrix of Pearson correlation coefficients was calculated for each pair of risk factors.

Multivariable Cox regression analyses were performed to assess the association of polygenic risk and different blood lipid levels with incident events of CAD. Blood lipids considered were TC, HDL, LDL-C, as well as TC:HDL and LDL-C:HDL ratios.

Individuals were divided into three groups based on their polygenic risk: Low PRS, Intermediate PRS, High PRS: <=10th, >10th<=90th, >90th percentile of PRS distribution, respectively. We binned Low PRS, Intermediate PRS, and High PRS populations according to different ranges for each blood lipid and calculated for each bin the Hazard ratio (HR) relative to the Intermediate PRS with Optimal blood lipid levels, used as reference populations. Optimal blood lipid levels were as follows: TC: below 200 mg/dL; LDL-C: below 130 mg/dL; HDL: above or equal to 80 mg/dL; TC:HDL: below 3.0; LDL-C:HDL: below 2.0.

In order to assess for possible interactions between LDL-C and PRS and between HDL and PRS, a Cox regression analysis was performed with quantitative scaled PRS, scaled LDL-C, scaled HDL, LDL-CxPRS and HDLxPRS as variables.

All Cox regression analyses were adjusted for age at recruitment, gender, genotyping array, the first four principal components of ancestry, SBP, family history of heart disease, smoking and diabetes status, TDI, BMI, HbA1C, TG, CRP, and DIG.

The DIG variable has been modelled as an integer variable ranging from 0 to 10, reflecting the number of dietary intake goals achieved by each participant. UK Biobank fields used to model the DIG variables were taken from Said et al. (2018). To maximize the likelihood of reporting true findings, a P value threshold was set for significance at 0.005 instead of 0.05 (Benjamin et al., 2018).

### Results

### Predictive Performances of CAD PRSs

The SCT algorithm identified a set of 291,969 SNPs that best predicted the onset of CAD in the validation dataset (see FIG. 1).

Since SCT identified a smaller number of SNPs than PRSs from Khera (Khera et al., 2018; 6,630,150 SNPs) and Inouye (Inouye et al., 2018; 1,745,180 SNPs), the Applicant assessed if the additional SNPs in these two PRSs may have residual predictive power in combination with STC-selected SNPs. The Applicant therefore generated and tested two additional PRSs by combining PRS from Khera et al. with SCT (SCT-K; 6,699,370 SNPs) and Inouye et al. with SCT (SCT-I; 1,920,136 SNPs).

Table 1 shows that the new SCT PRS displayed higher predictive performance than PRSs from Khera et al. and Inouye et al.

Moreover, while the addition of the SNPs from Khera PRS to SCT (i.e., SCT-K PRS) didn't result in any significant improvement in predictive performances over SCT alone, the combination of SCT and Inouye SNPs (SCT-I PRS) generated a new PRS with the highest predictive performances. We thus used the SCT-I PRS in the following analyses.

### Odds Ratio of PRS and Blood Lipid

The Applicant next examined the ability of SCT-I PRS to stratify the risk of CAD in the UK Biobank population and compared that with those of blood lipids and associated (Apo)lipoproteins recorded at baseline. In order to avoid reverse causation, only incident cases of CAD were considered.

The target population used for the analysis (Baseline characteristics are provided in Table 2) was generated from the testing dataset, by excluding prevalent CAD cases, as well as all individuals with missing data on TC, HDL, LDL-C, ApoA, ApoB, Lp(a), SBP, HbA1c, TG, BMI, CRP, and DIG at baseline (see FIG. 1).

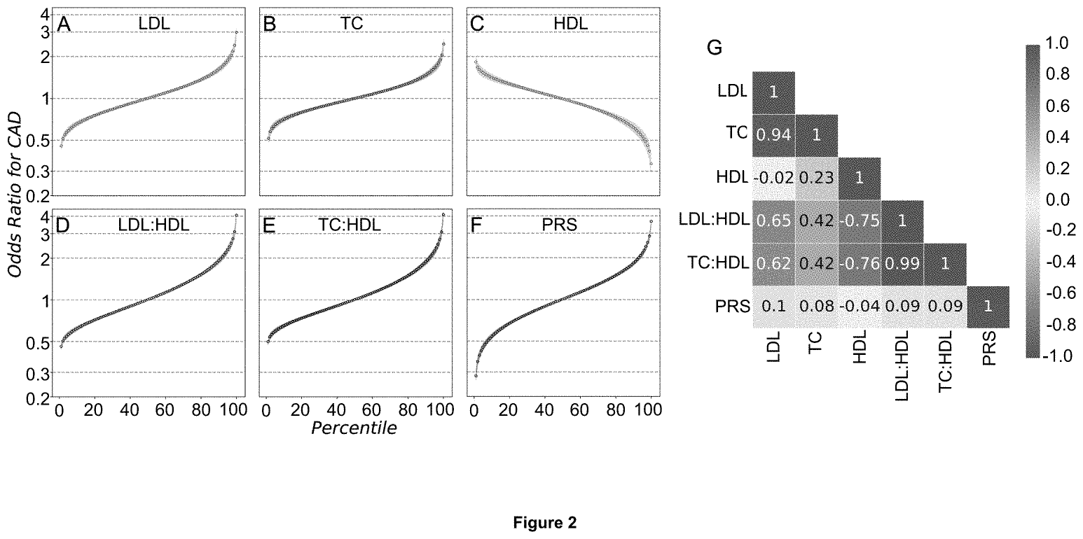

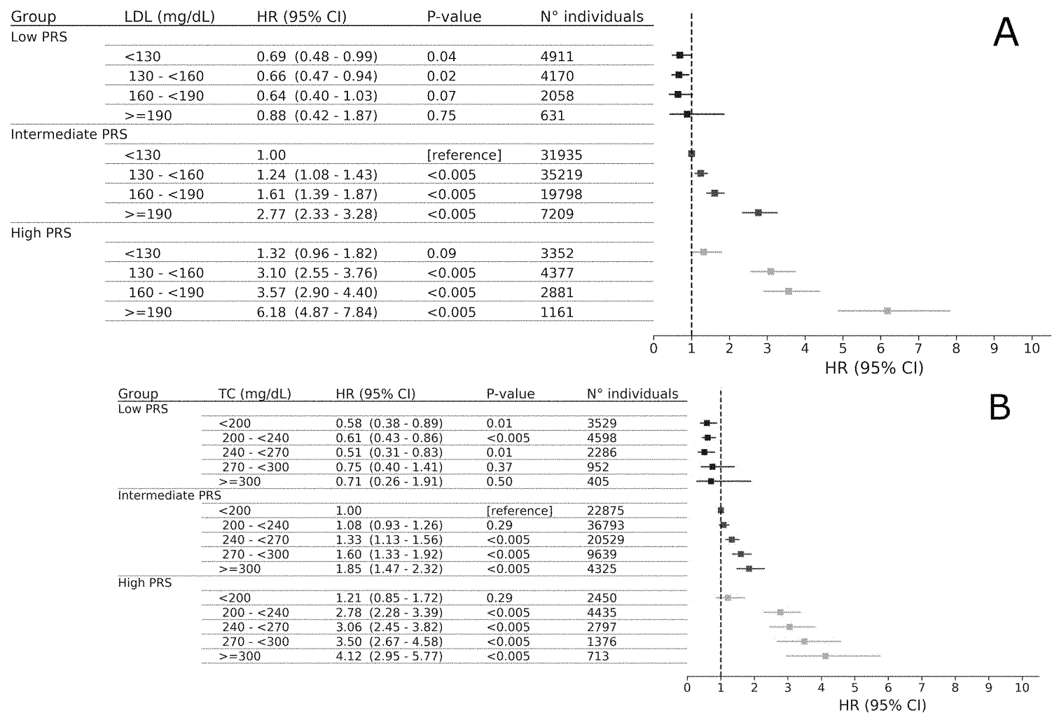

FIG. 2 shows the odds ratio per percentile of blood lipid and PRS distributions, computed respect to the average of each distribution.

Lipid ratios (TC:HDL and LDL-C:HDL) as well as SCT-I PRS displayed wider odds ratio ranges than single blood lipids alone (TC, LDL-C, and HDL): odds ratio for TC:HDL, LDL-C:HDL, and PRS ranged respectively from 0.50 to 4.12, from 0.46 to 4.07, and from 0.28 to 3.56, across percentiles of the corresponding distributions. On the other hand, odds ratio for TC, LDL-C, and HDL displayed narrower ranges, from 0.52 to 2.46, from 0.46 to 2.99, and from 1.83 to 0.34, respectively.

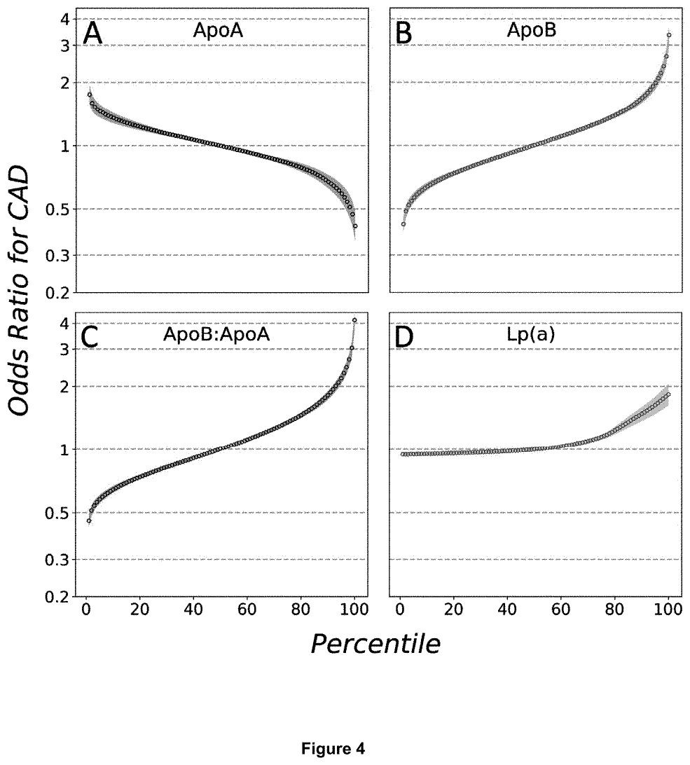

Similar results were obtained for (Apo)lipoproteins (see FIG. 4), where the odds ratio range of ApoB:ApoA (from 0.46 to 4.15) was wider than that of ApoB (from 0.42 to 3.35) and ApoA (from 1.76 to 0.41) alone. Finally, Lp(a) odds ratio range was the least observed (from 0.95 to 1.83).

Notably, among the factors with wider odds ratio range, TC:HDL, LDL-C:HDL, ApoB:ApoA, and PRS, the latter generated the wider range, with a 13 fold increase in odds ratio between the bottom and the top percentiles of the distribution, compared to a 8.25, 8.82, and 9.05 fold increase for TC:HDL, LDL-C:HDL, and ApoB:ApoA, respectively.

### Correlation Between PRS and Traditional Risk Factors

The Applicant then investigated if PRS and traditional risk factor distributions are correlated among individuals of the target population.

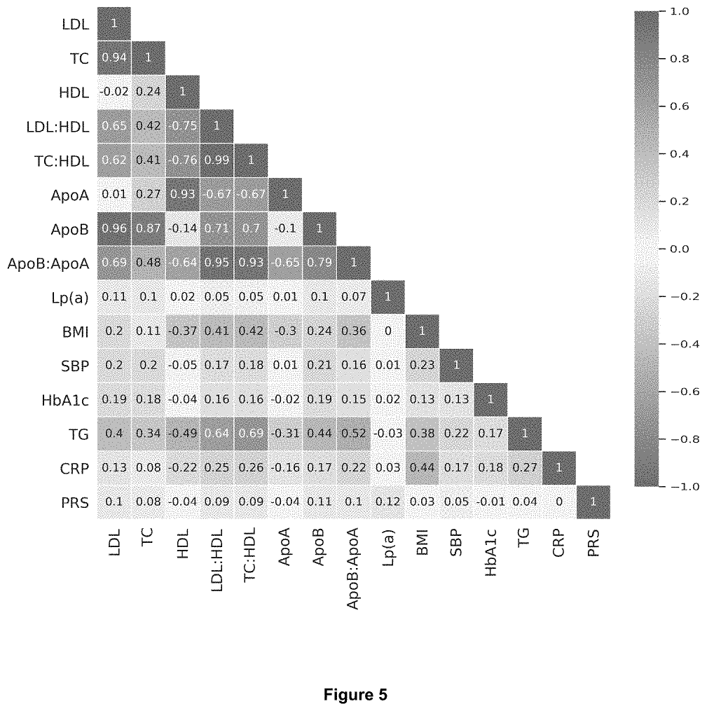

FIG. 5 displays the correlation matrix. Correlation coefficients displayed high (from ±0.50 to ±1.00) to moderate (from ±0.30 to ±0.49) degrees of correlation when any pair of blood lipids distributions were involved, the only exception being the low correlation between LDL-C and HDL. Most notably, the correlation coefficients between PRS and any blood lipids were very low, ranging from −0.04 for PRS with HDL to 0.1 for PRS to LDL-C. This finding indicates that there is a very low degree of overlap between individuals stratified through PRS and through any blood lipids, suggesting that PRS and blood lipids capture different components of the CAD risk.

Similar low values of correlation for PRS were obtained when additional risk factors are compared, such as ApoA, ApoB, ApoB:ApoA ratio, Lp(a), BMI, and SBP, HbAlC, TG, and CRP (see FIG. 5). CRP was moderately correlated only with BMI (0.44), possibly being related to inflammation processes in overweight individuals (Visser et al. 1999). TG were moderately correlated with each lipid, apolipoprotein and BMI, and highly correlated with their ratios (TC:HDL=0.69, LDL:HDL=0.64, ApoB:ApoA=0.52).

Association of Lipid Levels and Polygenic Risk with Incident CAD

PRS and blood lipids were orthogonal, capturing different independent components of CAD risk. It was of interest to investigate how the interplay between these two orthogonal sources contribute to the overall CAD risk in the population.

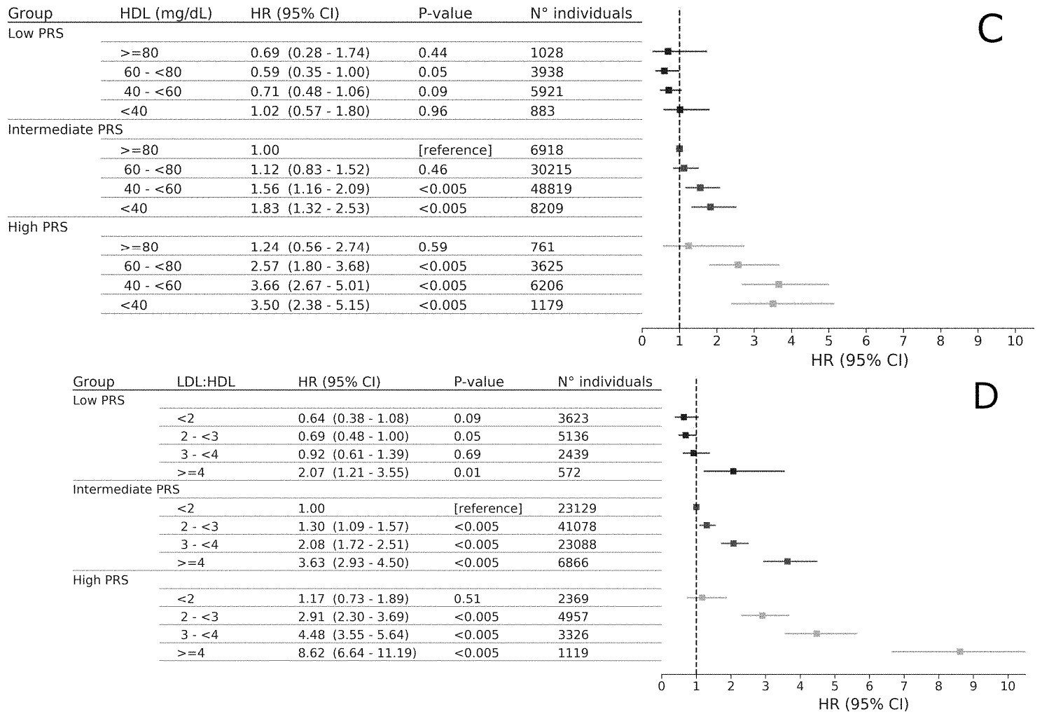

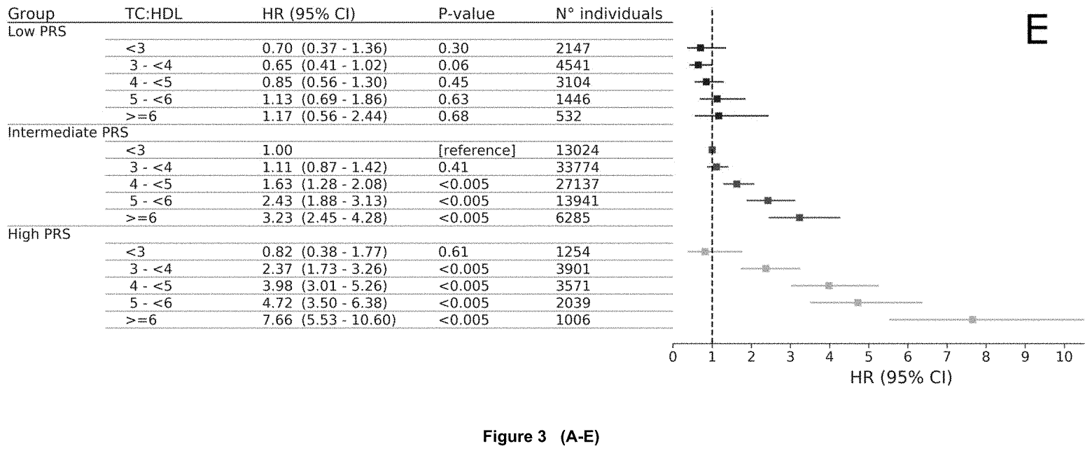

FIG. 3 illustrates the results of a Cox regression analysis testing the association of different lipid levels with incident CAD in three populations: individuals with a high polygenic risk (High PRS: individuals at the top 10 percentiles of PRS distribution), individuals with intermediate polygenic risk (Intermediate PRS: between the bottom 10 and the top 10 percentiles of PRS distribution), and individual with low polygenic risk (Low PRS: below the bottom 10 percentiles of the PRS distribution).

For LDL-C(FIG. 3A) above 130 mg/dL, HRs increase for higher levels, with HRs of High PRS being at least two fold higher than those of Intermediate PRS. Below the 130 mg/dL threshold, HR in High PRS group was not statistically significantly higher compared to Intermediate PRS with the same LDL-C level. High PRS with borderline LDL-C(130-159 mg/dl) group showed higher HR (3.10; 95% CI, 2.55-3.76) than Intermediate PRS with very high LDL-C(>190 mg/dl; HR 2.77; 95% CI, 2.33-3.28). On the other hand, in the Low PRS group the highest LDL-C level (>190 mg/dl) did not show statistically significant increased risk compared to optimal LDL-C level (<130 mg/dl) in Intermediate PRS. Notably, relative risk reduction between Very High and Optimal LDL-C levels were lower in Low PRS (45%) compared to High PRS (618%) and Intermediate PRS (277%).

Similar behavior was observed for TC (FIG. 3B): In High PRS, TC levels displayed a positive association with CAD for values above 200 mg/dL, while there was not significant association at TC<200 mg/dL. Incident CAD risk was inversely associated with HDL levels (FIG. 3C). In High PRS, a positive association with incident CAD was observed for HDL levels below 80 mg/dL. In High and Intermediate PRS, Lipid ratios (LDL-C:HDL and TC:HDL in FIGS. 3D and 3E, respectively) showed a positive association with CAD for values above 2.0 and 3.0 for LDL-C:HDL and TC:HDL, respectively. In High PRS, lipid ratios displayed a much stronger positive association with incident CAD compared to single lipid levels. Despite this, in High PRS individuals Optimal values of lipid ratios (below 2.0 and 3.0 for LDL-C:HDL and TC:HDL, respectively) displayed not statistically significant HRs compared to Intermediate PRS with the same Optimal values. Very High LDL-C:HDL were positively associated with CAD incidence even in the Low PRS group (HR 2.07; 95% CI, 1.21-3.55).

Interactions Between LDL-C, HDL and PRS with Incident CAD

In order to assess for possible interactions between LDL-C and PRS and between HDL and PRS, a Cox regression analyses was performed with quantitative scaled PRS, scaled LDL-C, scaled HDL, LDL-CxPRS and HDLxPRS as variables controlling for lifestyle and clinical factors and biomarkers associated with CAD incidence (details on these aspects have been illustrated above). According to a P value threshold for significance of 0.005, the Applicant identified significant interactions between LDL-C and PRS (HR: 1.07, P value: <0.005, see Table 4), but not between HDL and PRS (HR: 0.94 P value: 0.02, see Table 4).

The Applicant next assessed the HRs of PRS, LDL-C and their interaction (LDL-CxPRS) excluding from the model the non-significant interaction between PRS and HDL (HDLxPRS). PRS showed higher HR per SD (1.45; 95% CI, 1.39-1.53) than LDL-C(HR 1.39; 95% CI, 1.32-1.45) and their interaction (LDL-CxPRS) had a p-value lower than 0.005 (see Table 5).

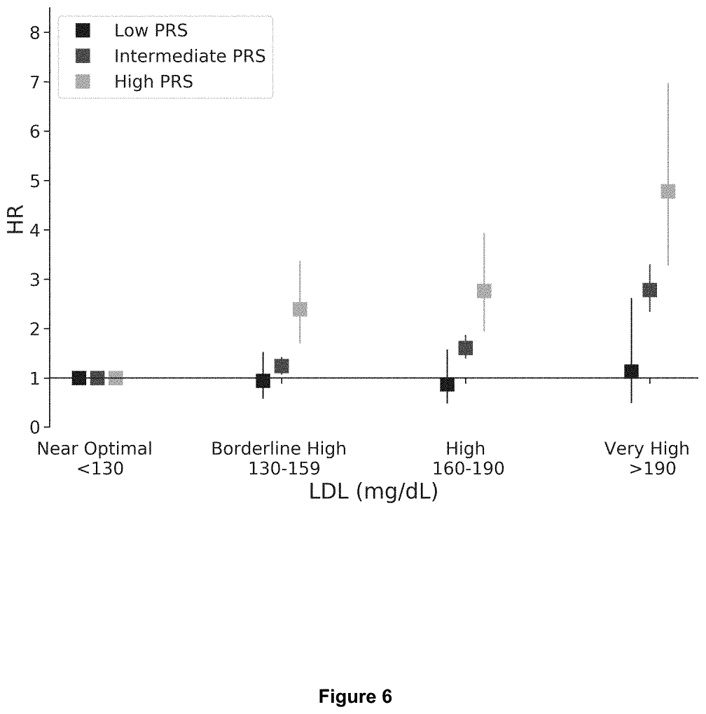

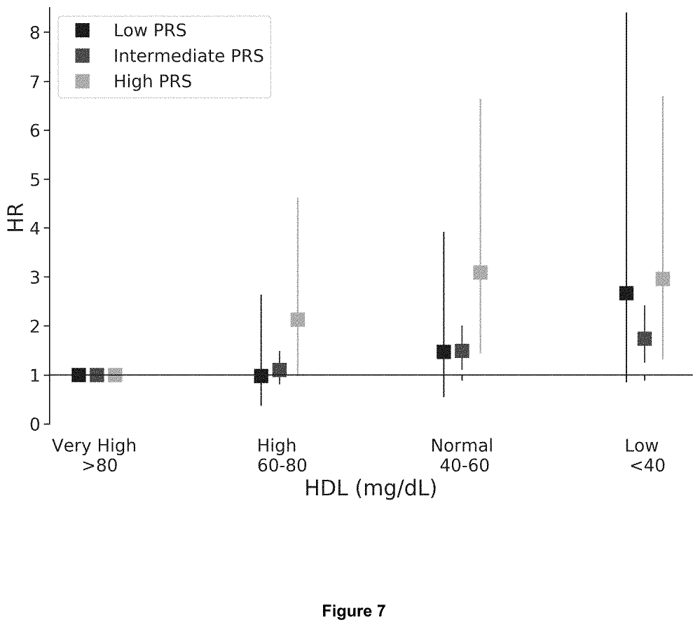

In order to explore the effect of the above findings, the association with incident CAD was assessed for different levels of LDL-C and HDL in each polygenic risk group separately, taking as reference Optimal LDL-C(<130 mg/dL) and Very High HDL (>80 mg/dL) (see FIG. 6 and FIG. 7), respectively. While for LDL-C, High PRS group showed much steeper increase in HR than Low PRS and Intermediate PRS groups for increasing LDL-C, for HDL the polygenic risk groups showed not significantly increased HRs across HDL levels tested.

The following more detailed remarks are provided about the figures.

FIG. 2: Correlation Matrix and Odds Ratio for Incident CAD by PRS, Lipids and their Ratios).

Predicted odds ratio of incident CAD for each percentile of the distribution of the following CAD risk factors: LDL-C(Panel A), TC (Panel B), HDL (Panel C), LDL-C:HDL ratio (Panel D), TC:HDL ratio (Panel E), and SCT-I PRS (Panel F). Odds ratio were calculated through logistic regression models with percentiles-specific average values of PRS and risk factors as predictive variables and incident CAD outcomes as response variables. Panel G. Matrix of Pearson correlation coefficients highlighting the pairwise correlation between distributions of blood lipid concentrations and PRS score in the target dataset population.

### FIG. 3: Risk of CAD Events in the UK Biobank Prospective Cohort According to Polygenic Risk and Blood Lipid Levels.

The population of the target dataset was divided in three groups: Individuals with Low (bottom 10 percentiles of PRS distribution), Intermediate (between the bottom 10 and the top 10 percentiles of PRS distribution), and High (above the top 10 percentiles of PRS distribution) PRS. These three groups were stratified according to different ranges of LDL-C(Panel A), TC (Panel B), HDL (Panel C), LDL-C:HDL (Panel D), and TC:HDL (Panel E) levels. For each blood lipid, HR were calculated by comparing each lipid level of Low, Intermediate, and High PRS groups relative to the reference population in a Cox proportional-Hazards model adjusted for age, gender, genotyping array, the first 4 principal components of ancestry, townsend deprivation index, diabetes and smoking status, family history of heart disease, systolic blood pressure, Glycated hemoglobin, Triglycerides, Body Mass Index, C-reactive protein, and dietary intake goals. For each lipid, the reference population corresponded to the first lipid level in the Intermediate PRS subpopulation. For each lipid level, the corresponding population size is reported (Number of Individuals). The vertical dashed line indicates the reference HR value of 1.

### FIG. 4: Odds Ratio for Incident CAD by Apolipoproteins.

Predicted odds ratio of incident CAD for each percentile of the distribution of the following CAD risk factors: ApoA (Panel A), ApoB (Panel B), ApoB:ApoA ratio (Panel C), and Lp(a) (Panel D). Odds ratio were calculated through logistic regression models with percentiles-specific average values of risk factors as predictive variables and incident CAD outcomes as response variables.

### FIG. 5: Correlation Matrix Between CAD Risk Factors.

Matrix of Pearson correlation coefficients highlighting the pairwise correlation between distributions of blood lipid concentrations and PRS score in the target dataset. TC: total cholesterol, HDL high-density cholesterol, LDL-C: low-density cholesterol, LDL-C:HDL: low-density over high-density cholesterol ratio, TC:HDL: low-density over high-density cholesterol ratio, ApoA: Apolipoprotein A, ApoB: Apolipoprotein B, Ln(a): Lipoprotein a, BMI: Body Mass Index, SBP: Systolic Blood Pressure, HbA1C: Glycated hemoglobin, TG: Triglycerides, CRP: C-reactive protein, PRS: SCT-I Polygenic risk score.

FIG. 6: Differential increased CAD risk by LDL-C in groups with different polygenic risk.

Increased risk Association of LDL-C with incident CAD in Low, Intermediate, and High PRS groups. In each group, HRs were calculated relative to the near optimal (<130 mg/dL) reference LDL-C level in a Cox proportional-Hazards model adjusted for age, gender, genotyping array, the first 4 principal components of ancestry, townsend deprivation index, diabetes and smoking status, family history of heart disease, systolic blood pressure, Glycated hemoglobin, Triglycerides, Body Mass Index, C-reactive protein, and dietary intake goals. FIG. 7: CAD risk by HDL in groups with different polygenic risk.

Association of HDL with incident CAD in Low, Intermediate, and High PRS groups. HRs were calculated relative to the very high (>80 mg/dL) reference HDL level in a Cox proportional-Hazards model adjusted for age, gender, genotyping array, the first 4 principal components of ancestry, townsend deprivation index, diabetes and smoking status, family history of heart disease, systolic blood pressure, Glycated hemoglobin, Triglycerides, Body Mass Index, C-reactive protein, and dietary intake goals.

### Discussions

Here, the Applicant analyzes the interplay between PRS and clinical blood lipids in determining the risk of incident CAD in the prospective cohort of the UK Biobank.

The analysis of the association of incident CAD with different lipid levels and the novel PRS (illustrated in this description) in three subpopulations at different polygenic risk (Low: bottom decile, Intermediate: 10th-90th percentiles and High: top decile) supports the following conclusions.

First, the Applicant has identified for the first time a significant interaction between polygenic risk and LDL-C in determining CAD outcome. The effect that LDL-C levels have on an individual's CAD risk depends on the polygenic background.

Second, the Applicant has found find that the combination of High PRS and Borderline LDL-C gives individuals an incident CAD risk that is greater than individuals with Intermediate or Low PRS but who have Very High LDL-C(>190 mg/dL).

Third, individuals with Optimal lipid levels do not show increased CAD risk despite their High polygenic risk.

Fourth, individuals in the lowest 10 percentiles of the PRS distribution are not at increased risk of CAD regardless of their LDL-C levels.

The above mentioned results have important implications for the primary prevention of CAD.

For example, according to current guidelines (Stone et al., 2014), individuals with Borderline LDL-C(130-159 mg/dL) will not be recommended statins. However according to the results here presented, individuals in this group who also have High polygenic risk have a greater relative risk of CAD than individuals with Intermediate or Low polygenic risk, but whose Very High LDL-C(>190 mg/dL) would lead to the recommendation of statins.

The Applicant's analysis also shows that the higher risk of incident CAD in High PRS individuals is reduced to that of the reference population if lipid levels are within the following conditions: <130 mg/dL for LDL-C, <200 mg/dL for TC, >80 mg/dl for HDL, and <2.0 and 3.0 for LDL-C:HDL and TC:HDL, respectively.

A prior study (Christiansen et al., 2020) identified a strong positive association between total plaque burden and a CAD PRS, with an increase of +78% of coronary artery calcium scores and a +16% of severe stenosis score per SD increase in PRS. Notably, the levels of LDL-C—that is the blood lipid mostly contributing to plaque formation (Bentzon et al., 2014)—were very similar in all polygenic risk groups, (average LDL-C: 120 mg/dL, 128 mg/dL, and 131 mg/dL in Low, Average, and High PRS, respectively). This is consistent with the finding of this study, suggesting that specific polygenic backgrounds may modulate the atherosceltric effect of LDL-C.

To the Applicant's knowledge, this is the first report that extensively compared the odds ratio range of a PRS (SCT-I) with a wide set of blood lipids and Apolipoproteins.

The Applicant observed that each lipid (and apolipoproteins) ratios generated wider odds ratio ranges across the percentiles of their distributions than single lipids (FIG. 2, FIG. 4). This agrees with previous work highlighting that TC:HDL and LDL-C:HDL display higher predictive performances for CAD than single lipids (Shai et al., 2004; Ingelsson et al., 2007). In addition, we showed that PRS on its own has a greater odds ratio range than any individual blood lipid and their ratios (FIG. 2).

The Applicant also reported for the first time the association of lipid levels to incident CAD in groups with different polygenic risk.

However similarly to the analyses reported here, two previous studies (Khera et al., 2016; Said et al., 2018) analyzed the interplay between different polygenic backgrounds with a modifiable parameter: lifestyle (poor/unfavorable, intermediate, and ideal/favorable).

In both studies, lifestyle was modelled as an aggregated categorical variable comprising smoking status, BMI, frequency of physical activity, and ideal or poor diet. These studies and the results presented here share similar patterns of interplay between the polygenic background and the additional factor considered. That is, they all observe a monotonic increase of CAD association (HRs) with increasing polygenic risks and the additional factor (be it lifestyle or lipid levels).

However, there are two notable differences between this study and those mentioned above.

First, in this analysis even in individuals at High polygenic risk, Optimal lipid level thresholds show non-significant increased risk compared to Intermediate polygenic risk with Optimal blood lipids. This is not observed in either Khera et al. (2016) or Said et al. (2018), where the CAD risk in High PRS individuals is never completely reduced in favorable lifestyles categories.

Second, Said et al. (2018) found no significant interaction between lifestyle and genetic risk with CAD. In contrast, the Applicant's study, here reported, has identified a statistically significant interaction between PRS and LDL-C in modulating individual's risk of CAD.

A previous study (Hindy et al., 2018), analyzing the association of smoking status to incident CAD in different polygenic backgrounds (lower, intermediate, and upper quartiles of PRS distribution) identified statistically significant interaction between smoking and PRS, highlighting the importance of considering the polygenic background in studying lifestyle effects on CAD risk.

Elliott and colleagues (2020) showed that a model using age, sex and PRS as covariates to predict CAD events had the same predictive power (C-statistics 0.76) of the Pooled Cohort Equation (PCE), a widely used model comprising several risk factors such as TC, HDL, age, sex, SBP, smoking, diabetes status and ethnicity (Yadlowsky et al. 2016).

PRS has the advantage to be detected early in life, as opposed to conventional risk factors used in the PCE that become relevant at mid and late age. Therefore PRS can allow the implementation of preventive strategies earlier in life. The potential efficacy of early intervention in the primary prevention setting is further supported by the recent evidence (Ference et al. 2019) that a lifelong genetic exposure to 38.67-mg/dL lower LDL-C and 10-mm Hg lower SBP was associated with a 4.5 fold risk reduction of major coronary events.

The finding of the Applicant's study that increased CAD risk conferred by LDL-C is enhanced by the polygenic risk, indicates that individuals with High polygenic risk could benefit the most from lifetime exposure to lower LDL-C levels, warranting the consideration of early detection of both PRS and LDL-C in the general population.

### Strengths and Limitations

Major strengths of this study were: the development and application of a new CAD PRS with higher predictive performance than previously published CAD PRS, a large prospective cohort of about 120 000 individuals, as well as the adjustment for lifestyle (smoking, diet intake goal, BMI), clinical (diabetes, blood pressure, family history), biomarkers (Glycated hemoglobin, Triglycerides, C-reactive protein) and various demographic (TDI) confounders.

This study has limitations. First, lipid levels used in the analyses are those reported at baseline. Therefore, changes in lipid levels over time were not considered in the present analyses. Second, the analysis has been performed on a population of white British individuals. This has implications on the generalizability of the reported results on different ethnic groups or cohorts. Third, the association of different lipid levels with CAD risk cannot be taken as a causal relationship, since lipid levels status were not randomized. Therefore, future research is required to confirm reported results on additional cohort populations and to address the causal relationship between achieving lower lipid levels and reduced CAD risk in the High polygenic risk group.

### Conclusions

In conclusion, physiological parameters, such as blood lipid levels, together with polygenic background, represent two independent contributions to the overall risk of incident CAD. Risk of incident CAD conferred by high LDL-C level was modified by the polygenic risk. However for various lipids, lipid levels at Optimal thresholds showed completely counterbalanced CAD risk despite a High polygenic background. The tailoring of lipid guidelines on the basis of the polygenic background seems a promising strategy to reduce the incidence of CAD in the general population.

An individual's lifetime risk of Coronary Artery Disease (CAD) is determined by a combination of genetic and lifestyle factors. Whilst adherence to a healthy lifestyle can help individuals with high genetic risk reduce their lifetime risk of CAD, the extent to which blood lipid levels affect CAD risk in individuals with varying genetic risk remains unknown.

To explore how genetics, blood lipids and CAD risk interact, a novel genome-wide polygenic risk score (PRS) for CAD was derived, and has been illustrated in this disclosure. Then, the PRS was applied to individuals from the UK Biobank and such individuals were divided them into Low PRS (bottom 10 percentiles of PRS distribution), Intermediate PRS (PRS in the 10th-90th percentiles), and High PRS (top 10 percentiles), and further stratified individuals by blood lipid levels.

### Summary Results

It has been found that the elevated CAD risk conferred by high low-density lipoprotein cholesterol (LDL-C) was modified by the interaction with PRS (P-value interaction: <0.005). Individuals with High PRS and whose LDL-C was Borderline (between 130 and 160 mg/dL) had higher CAD relative risk (HR 3.10; 95% CI, 2.55-3.76) than those at Intermediate PRS whose LDL-C was Very High (>190 mg/dL; HR 2.77; 95% CI, 2.33-3.28).

Furthermore, individuals with High PRS but whose lipid levels were below the following thresholds did not have a significantly increased risk for incident CAD: LDL-C<130 mg/dL, total Cholesterol (TC)<200 mg/dL, LDL-C:HDL<2.0 and TC:HDL<3.0.

In addition, individuals with Low PRS and Very High LDL-C(>190 mg/dl) did not have increased CAD risk, which was comparable to individuals with Intermediate PRS and Optimal LDL-C(<130 mg/dL).

The results reported in this description have important implications for the primary prevention of coronary artery disease.

Currently, healthy individuals with Borderline LDL-C(130-159 mg/dL) are not considered to be at high risk of CAD. Here we demonstrate that the combination of Borderline LDL-C and High PRS results in CAD relative risk which is greater than individuals without high polygenic risk, but whose LDL-C levels are high enough for statins to be recommended (>190 mg/dL).

This analysis therefore demonstrates that PRS can identify a proportion of the population who are at high-risk of CAD but who are invisible to current approaches for assessing CAD risk.

Moreover, of perhaps greater significance is the evidence that individuals who have a combination of High PRS and Optimal blood lipid levels do not have greater risk of CAD than individuals without high polygenic risk and the same Optimal blood lipid levels.

The results reported above suggest that high polygenic risk for CAD could be overcome by controlling blood lipid levels.

The Applicant proposes that incorporating PRS into CAD risk assessment early in life could allow individuals at high polygenic risk to benefit from tailored blood lipid guidelines and avoid lifetime exposure to potentially damaging PRS-dependent LDL-C levels.

### Tables

TABLE 1
Predictive performance of PRSs for CAD assessed in this study.

| PRS panel | SNPs in PRS | AUC (95% Cl) |
|---|---|---|
| Khera | 6630150 | 0.811 (0.802-0.820) |
| Inouye | 1745180 | 0.812 (0.803-0.822) |
| SCT | 291969 | 0.814 (0.806-0.822) |
| SCT + Khera (SCT-K)* | 6699370 | 0.814 (0.806-0.823) |
| SCT + Inouye (SCT-I)* | 1920136 | 0.817 (0.809-0.826) |

Khera: PRS developed by Khera et al. (2018). Inouye: PRS developed by Inouye et al. (2018).
SCT: PRS for CAD developed with the SCT algorithm.
SCT + Khera (SCT-K): combination of SCT and Khera PRSs.
SCT + Inouye (SCT-I): combination of SCT and Inouye PRSs.
*For SNPs present in both SCT and a second panel (Khera or Inouye), effect sizes from SCT were taken.
AUC (95% Cl): Area Under the ROC Curve and 95% confidence intervals.

TABLE 2
Baseline characteristics of the target population.

| | | | | | | | Intermediate | | | |
|---|---|---|---|---|---|---|---|---|---|---|
| Characteristics | Males | | Females | | Low PRS | | PRS | | High PRS | |
| Total, N° (%) | 49476 | (42.03) | 68226 | (57.9) | 11770 | (10) | 94161 | (80) | 11771 | (10) |
| Age, mean (SD), y | 55.15 | (8.19) | 55.43 | (7.98) | 55.93 | (7.98) | 55.33 | (8.08) | 54.52 | (8.07) |
| CAD cases, N° | 1462 | (2.96) | 588 | (0.86) | 93 | (0.79) | 1542 | (1.64) | 415 | (3.53) |
| (%) | | | | | | | | | | |
| LDL-C, mean | 144.61 | (29.55) | 143.66 | (31.85) | 138.31 | (30.16) | 144.17 | (30.81) | 148.93 | (31.55) |
| (SD), mg/dL | | | | | | | | | | |
| TC, mean (SD), | 224.48 | (38.28) | 231.40 | (40.99) | 222.37 | (39.29) | 222.36 | (39.9) | 233.54 | (40.81) |
| mg/dL | | | | | | | | | | |
| HDL, mean | 50.81 | (11.63) | 62.66 | (13.83) | 58.82 | (14.49) | 57.66 | (14.19) | 56.69 | (14.01) |
| (SD), mg/dL | | | | | | | | | | |
| LDL-C:HDL, | 2.98 | (0.85) | 2.41 | (0.77) | 2.50 | (0.81) | 2.65 | (0.85) | 2.78 | (0.87) |
| mean (SD) | | | | | | | | | | |
| TC:HDL, mean | 4.60 | (1.12) | 3.84 | (0.98) | 3.97 | (1.05) | 4.16 | (1.11) | 4.32 | (1.13) |
| (SD) | | | | | | | | | | |
| ApoA, mean | 1.44 | (0.22) | 1.65 | (0.26) | 1.58 | (0.27) | 1.56 | (0.27) | 1.54 | (0.27) |
| (SD), g/L | | | | | | | | | | |
| ApoB, mean | 1.08 | (0.22) | 1.05 | (0.23) | 1.02 | (0.22) | 1.06 | (0.23) | 1.10 | (0.23) |
| (SD), g/L | | | | | | | | | | |
| ApoB:ApoA, | 0.77 | (0.20) | 0.66 | (0.19) | 0.66 | (0.19) | 0.70 | (0.20) | 0.74 | (0.21) |
| mean (SD) | | | | | | | | | | |
| Lp (a), mean | 42.54 | (48.39) | 44.28 | (49.16) | 33.35 | (40.12) | 42.86 | (48.24) | 59.23 | (57.24) |
| (SD), nmol/L | | | | | | | | | | |
| Smoking, N° | 5598 | (11.31) | 5015 | (7.35) | 984 | (8.36) | 984 | (9.00) | 11.59 | (9.84) |
| (%) | | | | | | | | | | |
| Family history, | 18205 | (36.80) | 29490 | (43.22) | 4125 | (35.05) | 37978 | (40.33) | 5592 | (47.51) |
| N° (%) | | | | | | | | | | |
| SBP, mean | 139.44 | (16.98) | 133.13 | (18.68) | 133.98 | (17.77) | 135.83 | (18.26) | 137.19 | (18.56) |
| (SD) mmHg | | | | | | | | | | |
| Diabetes, N° | 266 | (0.54) | 206 | (0.30) | 36 | (0.31) | 385 | (0.41) | 51 | (0.43) |
| (%) | | | | | | | | | | |
| BMI, mean | 27.04 | (3.80) | 26.28 | (4.66) | 26.38 | (4.21) | 26.60 | (4.33) | 26.78 | (4.49) |
| (SD), kg/m² | | | | | | | | | | |
| HbA1C, mean | 34.76 | (4.99) | 34.71 | (4.21) | 34.68 | (4.36) | 34.73 | (4.53) | 34.79 | (4.89) |
| (SD), mmol/mol | | | | | | | | | | |
| TG, mean (SD), | 169.92 | (95.56) | 130.10 | (69.35) | 141.11 | (79.30) | 147.01 | (83.81) | 151.19 | (87.11) |
| mg/dL | | | | | | | | | | |
| CRP, mean (SD), | 2.25 | (4.10) | 2.40 | (4.04) | 2.28 | (4.15) | 2.34 | (4.02) | 2.41 | (4.32) |
| mg/L | | | | | | | | | | |

TABLE 3
List of UK Biobank fields and codes defining CAD outcomes
Coronary Artery Disease (CAD)

| UK Biobank | | |
|---|---|---|
| Field | Description | Codes |
| 20004 | Self Reported Operation | 1070, 1095, 1523 |
| | code | |
| 20002 | Self-reported non-cancer | 1075 |
| | illness | |
| 6150 | Vascular/heart problems | 1 |
| | diagnosed by doctor | |
| 41270 | Diagnoses - ICD10 | I21X, I22X, I23X, |
| | | I24X, I252 |
| 40001 | Underlying (primary) | I21X, I22X, I23X, |
| 40002 | cause of death: ICD10 | I24X, I252 |
| | Contributory (secondary) | |
| | causes of death: ICD10 | |
| 41272 | Operative procedures - | K401-K404, K411- |
| | OPCS4 | K414, K451-K455, |
| | | K491-K492 K498- |
| | | K499, K502, K751- |
| | | K754, K758-K759 |

TABLE 4
Interactions between LDL-C and PRS and between HDL and PRS

| | HR(95% Cl) | P value |
|---|---|---|
| PRS | 1.40 (1.33-1.48) | <0.005 |
| LDL-C | 1.44 (1.37-1.51) | <0.005 |
| HDL | 0.75 (0.70-0.80) | <0.005 |
| PRS × LDL-C | 1.07 (1.03-1.12) | <0.005 |
| PRS × HDL | 0.94 (0.90-0.99) | 0.02 |

Interactions between continuous scaled PRS and continuous scaled LDL-C and between PRS and continuous scaled HDL. HRs (95% CIs) refer to a 1 SD variable increase. HRs have been obtained in a Cox proportional-Hazards model adjusted for age, gender, genotyping array, the first 4 principal components of ancestry, townsend deprivation index, diabetes and smoking status, family history of heart disease, systolic blood pressure, Glycated hemoglobin, Triglycerides, Body Mass Index, C-reactive protein, and dietary intake goals.

TABLE 5
Interactions between LDL-C and PRS

| | HR(95% Cl) | P value |
|---|---|---|
| PRS | 1.45 (1.39-1.53) | <0.005 |
| LDL-C | 1.39 (1.32-1.45) | <0.005 |
| PRS × LDL-C | 1.06 (1.02-1.11) | <0.005 |

Interactions between continuous scaled PRS and continuous scaled LDL-C. HRs (95% CIs) refer to a 1 SD variable increase. HRs have been obtained in a Cox proportional-Hazards model adjusted for age, gender, genotyping array, the first 4 principal components of ancestry, townsend deprivation index, diabetes and smoking status, family history of heart disease, systolic blood pressure, Glycated hemoglobin, Triglycerides, Body Mass Index, C-reactive protein, and dietary intake goals.

### CITED REFERENCES

Abraham G, Malik R, Yonova-Doing E, Salim A, Wang T, Danesh J, Butterworth A S, Howson J M M, Inouye M, Dichgans M. Genomic risk score offers predictive performance comparable to clinical risk factors for ischaemic stroke. Nat Commun. 2019; 10: 5819.
Benjamin D J, Berger J O, Johannesson M, Nosek B A, Wagenmakers E J, Berk R, et al. Redefine statistical significance. Nat Hum Behay. 2018; 2: 6-10.
Bentzon J F, Otsuka F, Virmani R, Falk E. Mechanisms of plaque formation and rupture. Circ Res. 2014; 114: 1852-1866.
Bycroft C, Freeman C, Petkova D, et al. The UK Biobank resource with deep phenotyping and genomic data. Nature 2018; 562: 203-209.
Christiansen M K, Nissen L, Winther S, Møller P L, Frost L, Johansen J K, et al. Genetic Risk of Coronary Artery Disease, Features of Atherosclerosis, and Coronary Plaque Burden. J Am Heart Assoc. 2020; 9: e014795.
Cooney M T, Dudina A, De Bacquer D, Fitzgerald A, Conroy R, Sans S, et al. SCORE Investigators. How much does HDL cholesterol add to risk estimation? A report from the SCORE Investigators. Eur J Cardiovasc Prev Rehabil. 2009; 16: 304-314.
Ference B A, Graham I, Tokgozoglu L, Catapano A L. Impact of Lipids on Cardiovascular Health: JACC Health Promotion Series. J Am Coll Cardiol. 2018; 72:1141-1156.
Ference B A, Bhatt D L, Catapano A L, Packard C J, Graham I, Kaptoge S, Ference T B, Guo Q, Laufs U, Ruff C T, Cupido A, Hovingh G K, Danesh J, Holmes M V, Smith G D, Ray K K, Nicholls S J, Sabatine M S. Association of Genetic Variants Related to Combined Exposure to Lower Low-Density Lipoproteins and Lower Systolic Blood Pressure With Lifetime Risk of Cardiovascular Disease. JAMA. 2019; 322:1381-1391.
Fry A, Littlejohns T J, Sudlow C, Doherty N, Adamska L, Sprosen T, et al. Comparison of Sociodemographic and Health-Related Characteristics of UK Biobank Participants With Those of the General Population. Am J Epidemiol. 2017; 186: 1026-1034.
Hindy G, Wiberg F, Almgren P, Melander O, Orho-Melander M. Polygenic Risk Score for Coronary Heart Disease Modifies the Elevated Risk by Cigarette Smoking for Disease Incidence. Circ Genom Precis Med. 2018; 11: e001856.
Ingelsson E, Schaefer E J, Contois J H, McNamara J R, Sullivan L, Keyes M J, Pencina M J, Schoonmaker C, Wilson P W, D'Agostino R B, Vasan R S. Clinical utility of different lipid measures for prediction of coronary heart disease in men and women. JAMA. 2007; 298: 776-785.
Inouye M, Abraham G, Nelson C P, et al. Genomic risk prediction of coronary artery disease in 480,000 adults: Implications for primary prevention. J Am Coll Cardiol 2018; 72: 1883-1893.
Khera A V, Emdin C A, Drake I, et al. Genetic Risk, Adherence to a healthy lifestyle, and coronary disease. N Engl J Med 2016; 375: 2349-2358.
Khera A V, Chaffin M, Aragam K G, et al. Genome-wide polygenic scores for common diseases identify individuals with risk equivalent to monogenic mutations. Nat Genet 2018; 50: 1219-1224.
Lozano R, Naghavi M, Foreman K, et al. Global and regional mortality from 235 causes of death for 20 age groups in 1990 and 2010: a systematic analysis for the Global Burden of Disease Study 2010. Lancet 2012; 380: 2095-2128.
Mozaffarian D. Dietary and Policy Priorities for Cardiovascular Disease, Diabetes, and Obesity: A Comprehensive Review. Circulation. 2016; 133: 187-225.
Natarajan P, Young R, Stitziel N O, et al. Polygenic risk score identifies subgroup with higher burden of atherosclerosis and greater relative benefit from statin therapy in the primary prevention setting. Circulation 2017; 135: 2091-2101.
Nikpay M, Goel A, Won H H, et al. A comprehensive 1,000 genomes-based genome-wide association meta-analysis of coronary artery disease. Nat Genet 2015; 47: 1121-1130.
Privé F, Vilhjálmsson B J, Aschard H, et al. Making the most of Clumping and Thresholding for polygenic scores. Am J Hum Genet. 2019; 105: 1213-1221.
Said M A, Verweij N, van der Harst P. Associations of combined genetic and lifestyle risks with incident cardiovascular disease and diabetes in the UK Biobank study. JAMA Cardiol 2018; 3: 693-702.
Shai I, Rimm E B, Hankinson S E, Curhan G, Manson J E, Rifai N, Stampfer M J, Ma J. Multivariate assessment of lipid parameters as predictors of coronary heart disease among postmenopausal women: potential implications for clinical guidelines. Circulation. 2004; 110: 2824-2830.
Shai I, Rimm E B, Hankinson S E, Curhan G, Manson J E, Rifai N, Stampfer M J, Ma J. Multivariate assessment of lipid parameters as predictors of coronary heart disease among postmenopausal women: potential implications for clinical guidelines. Circulation. 2004; 110: 2824-2830.
Stone N J, Robinson J G, Lichtenstein A H, Bairey Merz C N, Blum C B, Eckel R H, et al. American College of Cardiology/American Heart Association Task Force on Practice Guidelines. 2013 ACC/AHA guideline on the treatment of blood cholesterol to reduce atherosclerotic cardiovascular risk in adults: a report of the American College of Cardiology/American Heart Association Task Force on Practice Guidelines. Circulation. 2014; 129: S1-S45.
Torkamani A, Wineinger N E, Topol E J. The personal and clinical utility of polygenic risk scores. Nat Rev Genet 2018; 19: 581-590.
Visser M, Bouter L M, McQuillan G M, Wener M H, Harris T B. Elevated C-reactive protein levels in overweight and obese adults. JAMA. 1999; 282:2131-5.
Yadlowsky S, Hayward R, Sussman J, McClelland R, Min Y, Basu S. Clinical Implications of Revised Pooled Cohort Equations for Estimating Atherosclerotic Cardiovascular Disease Risk. Ann Intern Med. 2018; 169:20-29.

1. A method for a predictive prognosis of onset of a cardiovascular disease in an individual, comprising the steps of:
acquiring or accessing data comprising the individual's low-density lipoprotein cholesterol level (LDL-Cp);
calculating at least one risk value (R), representative of a risk of onset of the cardiovascular disease in the individual, based on an operational value of a polygenic risk score (PRS) of the individual and an operational value of the individual's low-density lipoprotein cholesterol level (LDL-C), and also based on a first risk parameter (HR1) representative of a risk of onset of the cardiovascular disease induced by the polygenic risk score (PRS), of a second risk parameter (HR2) representative of a risk of onset of the cardiovascular disease induced by the low-density lipoprotein cholesterol level (LDL-C), and of a third risk parameter (HR3) representative of a risk of onset of the cardiovascular disease induced by the interaction of polygenic risk score (PRS) and low-density lipoprotein cholesterol level (LDL-C);
wherein said first risk parameter (HR1), said second risk parameter (HR2) and said third risk parameter (HR3) are calculated based on a pre-trained model and/or algorithm, to which said operational value of polygenic risk score (PRS) and said operational value of low-density lipoprotein cholesterol level (LDL-C) are provided in input;
determining and providing, as a prognostic result, an individualized risk value (Rp) of onset of the cardiovascular disease in an individual to be examined, based on said at least one calculated risk value (R);
wherein said step of calculating at least one risk value (R) comprises calculating a risk value (R) considering as the operational value of polygenic risk score (PRS) the value (PRSp) of the individual's polygenic risk score and as the operational value of the low-density lipoprotein cholesterol level (LDL-C) the individual's low-density lipoprotein cholesterol level (LDL-Cp); and
wherein the individualized risk value (Rp) of onset of cardiovascular disease corresponds to said calculated risk value (R).

2. A method according to claim 1, wherein the pre-trained model and/or algorithm comprises an algorithm trained in a preliminary training step,
wherein the preliminary training step comprises training the algorithm by machine learning and/or artificial intelligence techniques, based on known data.

3. A method according to claim 2, wherein the pre-trained model and/or algorithm comprises a regression model, which uses as covariates the polygenic risk score (PRS), low-density lipoprotein cholesterol level (LDL-C), combination of polygenic risk score and low-density lipoprotein cholesterol level (PRS×LDL-C); and
wherein the preliminary training step comprises training the regression model using as a reference dataset a first dataset of a population of individuals of which PRS and LDL-C are known, and for which the outcomes of onset of cardiovascular disease are known, for a first subset of individuals, or cases, or for which the non-onset of cardiovascular disease is known, after a predefined period of time, for a second subset of individuals or controls.

4. A method according to claim 3, wherein the pre-trained regression model is used to derive said first risk parameter (HR1), second risk parameter (HR2) and third risk parameter (HR3); and
wherein said preliminary training step comprises training the regression model with reference to said first risk parameter (HR1), said second risk parameter (HR2) and said third risk parameter (HR3) based on said reference dataset using as a dependent variable the cardiovascular disease onset or non-onset, obtainable from known data.

5. A method according to claim 3, wherein the pre-trained regression model is used to calculate said at least one risk value (R); and
wherein said preliminary training step comprises training the regression model with reference to said at least one risk value (R) based on said reference dataset using as a dependent variable the cardiovascular disease onset or non-onset, obtainable from known data.

6. A method according to claim 3, wherein the regression model is a Cox proportional hazards regression model.

7. A method according to claim 1, adapted to perform a predictive prognosis which takes into account one or more further risk factors;
wherein said step of calculating at least one risk value (R) comprises calculating the at least one risk value (R) also based on an operational value of each of one or more further covariates (COVx) associated with a respective one of said one or more further risk factors;
wherein the pre-trained model and/or algorithm is configured to calculate said first risk parameter (HR1), said second risk parameter (HR2) and said third risk parameter (HR3) also based on the operational values of each of said one or more further covariates (COVx); and
wherein the method further comprises accessing data of the individual to be examined, including individual values (COVxp) of each of said one or more further covariates associated with the one or more further risk factors.

8. A method according to claim 7, wherein the pre-trained model and/or algorithm comprises a regression model, which uses as covariates, in addition to the polygenic risk score (PRS) variable, a low-density lipoprotein cholesterol level (LDL-C) variable, a combination of polygenic risk score and low-density lipoprotein cholesterol level (PRS×LDL-C) variable, also said further variables (COVx) which encode the further risk factors; and
wherein the preliminary training step comprises training the regression model using as a reference dataset a second dataset of a population of individuals of which, in addition to PRS and CDC-L, also the individual values of each of said further covariates (COVx) is known.

9. Method according to claim 7, wherein said further risk factors comprise any sub-set of one or more of the risk factors belonging to the following set:
age, gender, genotyping array, the first 4 principal components of ancestry, Townsend Deprivation Index (TDI), diabetes and smoking status, family history of heart disease, systolic blood pressure (SBP), Body Mass Index (BMI), Glycated hemoglobin, HbA1C, Triglycerides (TG), C-reactive protein (CRP), and dietary intake goals (DIG); and/or
wherein the logistic regression is adjusted or conditioned based on any sub-set of one or more of the risk factors belonging to said set comprising age, gender, genotyping array, the first 4 principal components of ancestry, Townsend Deprivation Index (TDI), diabetes and smoking status, family history of heart disease, systolic blood pressure (SBP), Body Mass Index (BMI), Glycated hemoglobin, HbA1C, Triglycerides (TG), C-reactive protein (CRP), and dietary intake goals (DIG).

10. A method according to claim 1, wherein the risk value (R) is calculated based on a sum of products relating to different relevant variables, comprising at least said polygenic risk score (PRS) variable, low-density lipoprotein cholesterol level (LDL-C) variable, a combination of polygenic risk score and low-density lipoprotein cholesterol level (PRS×LDL-C) variable, wherein each product provides the multiplication of said first risk parameter (HR1), said second risk parameter (HR2) and said third risk parameter (HR3) for the difference between the operational value and the average value of the variables, respectively, polygenic risk score (PRS), low-density lipoprotein cholesterol level (LDL-C), combination of polygenic risk score and low-density lipoprotein cholesterol level (PRS×LDL-C).

11. A method according to claim 1, wherein the determined or calculated risk value is a relative risk value, with respect to an average risk referring to a population; or
wherein the determined or calculated risk value is an absolute risk value, relating to the individual, and representative of an individual's risk of developing cardiovascular disease within a certain period of time, or at a given age.

12. A method according to claim 1, wherein the cardiovascular disease is a coronary artery disease (CAD).

13. A device adapted to perform a predictive prognosis of the onset of a cardiovascular disease, comprising:
acquisition means for acquiring a quantity of blood of an individual and determining the individual's low-density lipoprotein cholesterol level (LDL-C) based on a test of the quantity of blood acquired;
an electronic interface, adapted to receive in input a calculated value of the individual's polygenic risk score (PRS);
a processor configured to receive said low-density lipoprotein cholesterol level (LDL-C) and polygenic risk score (PRS) value of the individual, and to perform a method for a predictive prognosis of the onset of cardiovascular disease according to claim 1,
wherein said acquisition and determination means, said electronic interface and said processor are comprised in a single portable device.

14. A method for providing a clinical evaluation or for deriving a therapeutic intervention, wherein said method is performed based on the results of a predictive prognosis method according to claim 1.
